(19) United States
(12) Patent Application Publication
(10) Pub. No.: US 2018/0100598 A1
(43) Pub. Date: Apr. 12, 2018

(54) NOVEL PINCH VALVE AND RELATED METHODS

(71) Applicant: Kenneth Pierson, Visalia, CA (US)

(72) Inventor: Kenneth Pierson, Visalia, CA (US)

(21) Appl. No.: 15/382,430

(22) Filed: Dec. 16, 2016

Related U.S. Application Data
(63) Continuation-in-part of application No. 15/289,065, filed on Oct. 7, 2016.

(51) Int. Cl. F16K 27/02; A61F 5/44; F16K 1/12; F16K 15/14

(52) U.S. Cl. CPC F16K 27/0209; A61F 5/4405; F16K 27/0254; F16K 15/148; F16K 1/12

(57) ABSTRACT

The present invention provides a novel pinch valve structure that allows for controlled dispensing of liquids, gases, and other flowable materials. The present invention address issues with current valves with an innovative structure that prevents leakage and spillage, and improves efficiency and ease of use. The pinch valves of the present invention may include a resilient compressible body with a distal drainage hole, a resilient actuator comprising at least two spring-like, resilient arms, a central piston member mechanically connected to the resilient arms, and a distal plug mechanically connected to the central piston member for engaging with and blocking the distal drainage hole.

### FIELD OF THE INVENTION

The present invention relates to a novel valve operated by compression of a resilient actuation mechanism and methods for using the same. More specifically, the embodiments of the present invention pertain to one-way pinch valves to regulate the release of liquids, gases, and other flowable materials from the valves.

### DISCUSSION OF THE BACKGROUND

Valves for dispersal of liquids (e.g., water coolers, faucets, beverage dispenser, nozzles, etc.), gases (e.g., oxygen tanks, carbon dioxide tanks, etc.), and other flowable materials (e.g., heterogeneous suspensions, slurries, etc.) are commonly used for beverage containers, waste reservoirs, medical containers, drug delivery packages, laboratory equipment, chemical processing tools, and several other settings. Such valves typically have clumsy or complicated mechanisms for operating the valve, such as the turning lever of a butterfly or ball valve, the coarse push-button actuation of a cross-valve, the awkward pulling motion for opening a pull-push cap valve (e.g., on a water bottle), etc. The manually operated actuators for such valves cannot be quickly or smoothly closed or opened (e.g., in fractions of a second).

Additionally, with regard to drainage valves for urostomy bags, other waste collection devices, and other medical devices suffer from being clumsy, as well. For example, urostomy bags typically have a stopcock valve or simply a snap-fit plug. Such valve structures make it difficult for a user to control the flow of urine or other waste when opening and draining the urine or other waste because the valves are clumsy and awkward and require two hands to operate. Therefore, the user may spill waste onto their hands or other undesirable places when trying to empty the receptacle when opening the snap-fit drainage valve.

Therefore, improved valve structures having more efficient and ergonomic designs that allow for quick and easy operation are desirable.

### SUMMARY OF THE INVENTION

The present invention is directed to embodiments of a novel one-way pinch valve that may be used for the dispersal of liquids, gases, and other flowable materials. The features of the present embodiments of the valve provide for quick and efficient actuation of the valve by the user, while preventing waste, leakage, and spillage. The novel pinch valves of the present invention may include a resilient compressible body with a distal drainage hole, a resilient actuator comprising at least two spring-like, resilient arms, a central piston member mechanically connected to the resilient arms, and a distal plug mechanically connected to the central piston member for engaging with and blocking the distal drainage hole.

The compressible outer body may have various shapes such as a bulb, polygonal prism, a cylinder, etc. The shape of the resilient compressible body may be selected to provide comfort and ease of manual use. The valves of the present invention may be used as a drainage or release valve for various liquid gas containers, such as waste containers (e.g., urostomy bags, incontinence collection bladders, etc.), beverage containers (e.g., beverage bottles, beverage bladders, water coolers, etc.), gas reservoirs (e.g., gas canisters, etc.), etc., and may allow a user to manually open the valve to drain or dispense liquid, gas, or other flowable material by simply pinching the compressible body of the valve to deform the actuator, such that the distal plug retracts from the distal drainage hole. Upon release of the valve, the actuator resiles to its original shape, quickly re-engaging the distal plug with the distal drainage hole due to the spring-like resilient construction of the actuator.

The valve body may be made of a sturdy, resilient polymeric material such as nitrile butadiene rubber (NBR), ethylene propylene diene monomer (EPDM) rubber, FKM fluoroelastomer, perfluoro-elastomers (FFKM), polytetrafluoroethylene (PTFE), hydrogenated nitrile butadiene rubber (HNBR), chlorinated polyvinyl chloride (CVPC), polypropylene (PP), polyvinyl chloride (PVC), or other polymer materials capable of forming flexible, resilient structures. In some embodiments, the resilient compressible body may be made of a transparent material (e.g., transparent polymer material). When the valve is made of a transparent material such as a polymeric rubber, one may easily see the actuator within the valve, allowing the user to engage the actuator by squeezing the compressible body at optimal positions on the resilient compressible body. In still other embodiments, the resilient compressible body may be made of an opaque material (e.g., an opaque rubber, vulcanized rubber, PTFE, etc.). In embodiments where the resilient compressible body is opaque, markings and/or protrusions may be included on the exterior of the resilient compressible body (e.g., prominently colored shapes and/or raised areas, etc.) to indicate where the user should engage and pinch the compressible bulb to properly compress the actuator. In some embodiments, the compressible body may be made from or the interior thereof may be coated with a non-reactive material (e.g., polyurethane, PVC, Kalrez®, etc.). The non-reactive material may prevent deterioration of the interior of the valve and the actuator when subjected to corrosive materials.

The actuator of the valves of the present invention may be resilient, sturdy structure seated within the compressible body, and may be operable to retract the distal plug from the distal drainage hole to open the valve. The actuator has two configurations: a closed configuration and an open configuration. When the actuator is in a closed configuration, the distal plug is engaged with the distal drainage hole and creates a seal, preventing the release or leakage of liquid, gas, or other flowable material. When the actuator is in an open position, the distal plug is retracted proximally into the compressible body, thereby opening the distal drainage hole releasing the liquid, gas, or other flowable material. In some embodiments, and without limitation, the distal plug may include a distal guide pin that passes through the distal drainage hole, and has a sufficient length such that the distal guide pin cannot be fully retracted into the body of the valve and at least a portion always remains on the exterior side of the distal drainage hole. The distal guide pin may aid in keeping the distal plug aligned with the distal drainage hole.

The actuator may be made from a resilient material that is flexible but resiles to its original shape, such as a resilient polymer material (e.g., polyoxymethylene, PVC, acrylonitrile butadiene styrene [ABS], high-density polyethylene [HDPE], acrylonitrile styrene acrylate [ASA], polypropylene [PP], etc.) or resilient metal spring material (e.g., steel alloys such as high carbon alloys, oil-tempered low-carbon alloys, chrome silicon alloys, chrome vanadium allows, and stainless steel; or beryllium copper alloy, phosphor bronze, etc.). In some embodiments, the actuator may be made from a composite material that includes, e.g., an inner metal structure providing resilient spring action to the actuator and an outer layer of non-reactive materials as a coating (e.g., polyurethane, PVC, Kalrez®, etc.).

The actuator may have two or more arms radiating out from a central piston member. The two or more arms may be connected to the central piston member at a joint angle of less than 90° (e.g., in a range of about 45° to about 80°, or any value therein) and radiate out to contact the inner wall of the compressible bulb. The distal ends of the two or more arms (e.g., two, three, four, five, six, etc.) may contact the inner wall of the compressible bulb at compression points, such that as the user applies force to the outer side of the compressible bulb at the compression points, the two or more resilient arms are pressed inward toward the central piston member, and the joint angle between each resilient arm and the central piston member decreases (e.g., without limitation, from about 20° to about 70°). As a result, the central piston member moves proximally, and the distal plug may be disengaged from the distal drainage hole.

The two or more resilient arms may be radially spaced from one another in a manner that allows the user to easily compress the actuator. For example, in embodiments having two resilient arms, the arms may be radially spaced at or about 180° from one another, allowing the user to pinch the valve with a natural grip at opposing sides of the valve. In embodiments in which the actuator includes more than two arms (e.g., three, four, five, six, etc.), the arms may radiate out from the central piston member at or about at equal intervals (e.g., in the case of three arms, the arms may be separated by 120° on the central piston member) so as to provide as many effective compression points as possible. In some embodiments, the resilient arms may each have an “elbow” and lower arm structure, where the elbow contacts the inner wall of the compressible bulb at a compression point, and the lower arm runs distally along the inner wall of the compressible bulb. At least a portion of the “lower arm” may be in contact with the interior wall of the compressible bulb and may assist in keeping the actuator in position within the compressible bulb. In some embodiments, one or more of the resilient arms may include a plate (e.g., a circular plate, a polygonal plate, etc.) at its distal end where it contacts the compression point to increase the interfacing surface area between the arm and the inner wall of the compressible body. In some embodiments, the distal ends of the arms may be connected to a ring structure that sits adjacent to the interior perimeter of the compressible body and may be complementary to the interior perimeter of the compressible body (e.g., a circular ring, a polygonal ring, etc.). The ring structure may be in mechanical contact with the interior perimeter of the compressible body on a plane that is or is about perpendicular to the longitudinal axis of the valve. In some embodiments, the ring structure may be mechanically attached to the interior perimeter of the compressible bulb (e.g., into a complementary groove in the interior perimeter by adhesive, pressure-fitting, or other attachment mechanisms). In such embodiments, the user can apply pressure at multiple points along the ring structure to compress the body and the one or more resilient arms of the actuator.

Some embodiments of the valve of the present invention may include alternative configurations of the actuator. For example, and without limitation, the actuator may be configured such that the central piston member has a distal plug that is seated in the distal drainage hole from the outer side, and the plug moves distally to open the valve as the actuator arms are compressed. In such embodiments, the arms may be attached to the central piston member at an angle greater than 90° (e.g., in a range of about 100° to about 135°, or any value therein). The distal ends of the two or more arms may contact the inner wall of the compressible bulb at compression points that are proximal to the central piston member, such that as the user applies force to the outer side of the compressible bulb at the compression points, the two or more resilient arms are pressed inward toward the central piston member, and the joint angle between each resilient arm and the central piston member increases (e.g., without limitation, from about 110° to about 160°). As a result, the central piston member moves distally, pushing the distal plug out of the compressible body from its seat in the distal drainage hole and opening the valve. After the compressible valve is released, the actuator resiles back to its original shape so that the central piston member and the distal plug are pulled proximally with the distal plug reseating in the distal drainage hole, thereby sealing the valve.

In some embodiments there may be a distal plug guide in proximity to the distal drainage hole. The distal plug guide is operable to receive the distal plug and help to align it with the distal drainage hole in the event that distal plug is misaligned with respect to the distal drainage hole. The distal plug guide also provides additional surface area to increase the strength of the seal between the distal plug and the distal drainage hole. The distal plug guide may have a complementary shape to the distal plug. For example, the distal plug may have a distally tapering shape (e.g., conical, etc.) and the shape of the distal plug guide may be complementary thereto (e.g., a frustrum of a cone), where the interior of the distal plug guide is complementary in shape and size to the exterior of the distal plug. In some implementations, it may be integrally formed with the compressible body.

In some embodiments, the distal plug, the distal plug guide, and/or the distal drainage hole may be coated with a hydrophobic material (e.g., a material having a surface energy below about 30 dynes/cm). The hydrophobic material may help prevent leakage of a fluid through the distal plug and or distal drainage hole. The hydrophobic material may provide a better seal between the distal plug, the distal plug guide, and/or the distal drainage hole to prevent the escape of hydrophilic liquids. The hydrophobic material may be coated on the surface of the distal plug, the distal plug guide, and/or the distal drainage hole, and may be a perfluoroalkyl polymer, hydrophobic polyol, polyether polyol, amine-initiated polyol, polytetrafluoroethylene (PTFE), polymeric MDI, manganese oxide polystyrene (MnO₂/PS), zinc oxide polystyrene (ZnO/PS), precipitated calcium carbonate, or other hydrophobic coating material.

In other embodiments in which the pinch valve is intended to be used to dispense hydrophobic fluids (e.g., alkanes, alkenes, alkynes, aromatic hydrocarbons, etc.), a hydrophilic substance may be coated on the distal plug, the distal plug guide, and/or the distal drainage hole. The hydrophilic substance may help prevent leakage of a hydrophobic fluid through the distal drainage hole when the distal plug is in the closed position. The hydrophilic substance may provide a better seal between the distal plug, the distal plug guide, and/or the distal drainage hole to prevent the escape of hydrophobic liquids. The hydrophilic material may be a polyurethane resin, polyvinylpyrrolidone (PVP), polyacrylate, or other hydrophilic coating material.

In some embodiments, the compressible valve may have a proximal attachment mechanism (e.g., ribbing, threading, Storz connector, etc.) that allows for the attachment, detachment, and replacement of the valve, if necessary. In such embodiments, the compressible valve may be detachably connected to a liquid, gas, or other flowable material source (e.g., reservoirs, containers, tanks, etc.) and may be replaceable. In some implementations, the proximal attachment mechanism may be a series of rubber barbs may be used to secure the novel compressible valve into a connector (e.g., on a bladder or other liquid reservoir) having interior circumferential ribbing with which the barbs may engage, thereby providing a secure engagement of the valve and the connector. In other examples, the proximal attachment mechanism may be threading to be received by connector having complementary threading (e.g., on a tank for containing a liquid or gas). In such embodiments, the compressible valve may be removed (e.g., by pulling the valve out, unscrewing the valve, etc.) from the connector of the source of liquid, gas, vapor, or slurry.

In other embodiments, the compressible valve may be integrally formed with a container for a liquid, gas, or other flowable material. For example, the compressible valve may be integrally formed with a container for dispensing a beverage or other liquid (e.g., a waste reservoir, a bladder, beverage bottle, a carboy), which may include a separate filling port for refilling the container. In still further embodiments, the compressible valve may be an integrally formed part of a single use container. For example, the compressible valve may be be integrally formed with a liquid dispensing container, such as a contact solution bottle, an eye drops bottle, a white out bottle, etc. In other examples, the new compressible valve may be an integral part of a gas dispensing canister (e.g., pressurized air, helium, carbon dioxide, etc.).

In further embodiments, the one-way valve of the present invention may directed to a one-way, in-line check valve in a gas or fluid line, where the resilient actuator is positioned and anchored within the gas or fluid line adjacent to a plug receiver for a distal plug of the actuator, such that the check valve is closed when the distal plug is seated in the plug receiver. In such embodiments, the check valve may be opened when a the gas or fluid applies sufficient pressure (e.g., a predetermined threshold pressure, to which the actuator may be calibrated) to the distal end of the distal plug, thereby unseating the distal plug from the plug receiver and opening the one-way check valve. Once the pressure falls below the threshold pressure, the force of the compressed actuator reseats the distal plug in the plug receiver, thereby closing the one-way check valve.

In such embodiments, the actuator may have a construction as described above, where the actuator arms are anchored to the interior gas or fluid tube, e.g., by a mechanical feature, such as a ridge, an adhesive, or other mechanism to hold the actuator in position in the tube. The plug receiver may have a complementary shape to the actuator plug such that the gas or fluid line can be sealed when the distal plug of the actuator is engaged with the plug receiver.

Several embodiments are discussed below, but the example embodiments shall not to be interpreted as an exhaustive list. One with ordinary skill in the art will recognize that the scope of the present invention includes further variations and equivalents to the specific examples described herein.

In one aspect, the present invention relates to a valve comprising a resilient compressible body having a distal drainage hole; and a resilient actuator within the resilient compressible body comprising at least two resilient arms, wherein each of the at least two resilient arms contacts an interior wall of the resilient compressible body, a central piston member mechanically connected to the at least two resilient arms, wherein each of the at least two resilient arms is attached to the central piston member at an oblique angle with respect to the longitudinal axis of the central piston member; and a distal plug mechanically connected to a distal end of the central piston member, the distal plug being operable to be inserted into and retracted from the distal drainage hole. The valve may have a closed configuration and an open configuration, wherein in the closed configuration the resilient actuator is in a relaxed conformation with a first angle between each of the at least two resilient arms and the central piston member when no external force is applied to the resilient compressible body, and the distal plug is engaged with the distal drainage hole; and in the open configuration an external force is applied to the resilient compressible body and the at least two resilient arms, wherein the resilient actuator is in a compressed conformation with a second angle between each of the at least two resilient arms and the central piston member, the second angle being smaller than the first angle, and the distal plug is retracted from engagement with the distal drainage hole.

The valve may further include a proximal attachment mechanism operable to connect to a vessel containing a fluid. In some examples, the resilient compressible body has a distal plug guide adjacent to the distal drainage hole, and the distal plug guide may be operable to guide and position the distal plug in the distal drainage hole. The distal plug may have a distally tapering shape. In some examples, the distal plug may have a conical shape and the distal plug guide may have a shape that is complementary to the conical shape of the distal plug. In some examples, the at least two resilient arms each include an upper portion connected to the central piston member, lower portion, and a flexible elbow where the upper portion and the lower portion meet. In some examples, the at least two resilient arms may be mechanically connected to the interior surface of the compressible body. In some examples, the resilient actuator may include at least three resilient arms, wherein each of the at least three resilient arms is in mechanical contact with an interior surface of the resilient compressible body. In some examples, the surface of the distal plug includes a hydrophobic material or a hydrophilic material. In some examples, the interior surface of the resilient compressible body may be coated with a non-reactive material. In some examples, the resilient actuator may be coated with a non-reactive material.

In another aspect, the present invention may be directed to a valve comprising an resilient compressible body having a distal drainage hole; and an actuator within the resilient compressible body comprising at least two resilient arms, wherein each of the at least two resilient arms, and a distal plug mechanically connected operable to be inserted into and retracted from the distal drainage hole. The valve may have a closed configuration and an open configuration, wherein in the closed configuration the resilient actuator is in a relaxed conformation with a first angle between the at least two resilient arms when no external force is applied to the resilient compressible body, and the distal plug is engaged with the distal drainage hole; and in the open configuration an external force is applied to the resilient compressible body and the at least two resilient arms, wherein the resilient actuator is in a compressed conformation with a second angle between the at least two resilient arms, the second angle being smaller than the first angle, and the distal plug is retracted from engagement with the distal drainage hole.

In some examples, the valve may further comprising a proximal attachment mechanism operable to connect to a fluid conduit or reservoir. In some examples, the valve may further include a distal plug guide adjacent to the distal drainage hole operable to guide and position the distal plug in the distal drainage hole. The distal plug may have a distally tapering shape. The distal plug guide may have a shape that is complementary to the distally tapering shape of the distal plug. The resilient actuator may be coated with a non-reactive material. The interior of the resilient compressible body may be made from or coated with a non-reactive material. In some examples, the distal end of at least one of the at least two resilient arms may be in mechanical connection with an interior surface of the compressible body. In some examples, the at least two resilient arms includes an interfacing plate at a distal end thereof, the interfacing plate being in mechanical connection with the connection member. In some examples, actuator may further comprise a ring structure in mechanical connection with the at least two resilient arms and in mechanical contact with an interior of the compressible body. In some examples, the surface of the distal plug includes a hydrophobic material or a hydrophilic material. In some examples, the interior surface of the resilient compressible body may be coated with a non-reactive material. In some examples, the resilient actuator may be coated with a non-reactive material.

In another aspect, the present invention may be directed to a check valve comprising a resilient actuator positioned within tubing for passing a gas or fluid, the resilient actuator comprising at least two arms mechanically connected to an interior of the tubing; a central piston member mechanically connected to the at least two arms, wherein each of the at least two arms is attached to the central piston member at an oblique angle with respect to the longitudinal axis of the central piston member; and a distal plug mechanically connected to a distal end of the central piston member; and a plug receiver in the tubing for receiving the distal plug, wherein the distal plug being operable to be inserted into and retracted from the plug receiver. The valve may have a closed configuration and an open configuration, wherein in the closed configuration the resilient actuator is in a relaxed conformation with the distal plug engaged with the plug receiver; and in the open configuration the resilient actuator is in a compressed conformation with the distal plug is retracted from engagement with the plug receiver. The actuator may have a pre-determined threshold resistance, such that when a gas or liquid applies a pressure equal to the pre-determined threshold resistance, the distal plug is unseated from the plug receiver and the check valve is opened.

In some examples, the distal plug may have a distally tapering shape. In some examples, the distal plug may have a conical shape and the distal plug guide may have a shape that is complementary to the conical shape. The at least two resilient arms may be mechanically connected to the interior surface of the tubing. The at least two arms may be mechanically connected to the interior surface of the compressible body. In some examples, the actuator may include at least three arms, wherein each of the at least three arms is in mechanical contact with an interior surface of the tubing. In some examples, the actuator may be coated with a hydrophobic material or a hydrophilic material. In some examples, the actuator may be coated with a non-reactive material.

In another aspect, the present invention may be directed to a method operating a valve comprising compressing a resilient compressible body having a distal drainage hole; and engaging a resilient actuator within the resilient compressible body by compressing at least two resilient arms, wherein each of the at least two resilient arms includes a flexible elbow and at least two resilient lower arms; raising at least one central piston member mechanically connected to the at least two resilient arms; and retracting a distal plug mechanically connected to the at least one central piston member operable to be inserted into and retracted from the distal drainage hole. The valve may be attached to a proximal attachment mechanism to a reservoir, container, or tank. The resilient compressible body may have a distal plug guide connected to the distal drainage hole guiding the distal plug in the distal drainage hole. The distal plug may have a conical shape creating a seal operable to prevent the release of a substance. The method may further comprise releasing the resilient compressible body to relax the resilient actuator; and retracting the distal plug and engaging the distal plug with the distal drainage hole.

In another aspect, the present invention may be directed to a method of manufacturing a valve comprising inserting resilient actuator into a resilient compressible body having a distal drainage hole, the resilient actuator comprising at least two resilient arms, wherein each of said at least two resilient arms contacts an interior wall of said resilient compressible body when inserted into said resilient compressible body; a central piston member mechanically connected to said at least two resilient arms, wherein each of said at least two resilient arms is attached to the central piston member at an oblique angle with respect to the longitudinal axis of the central piston member; and a distal plug mechanically connected to a distal end of said central piston member, wherein said distal plug is inserted into said distal drainage hole and is operable to be inserted into and retracted from said distal drainage hole. The valve may have a closed configuration and an open configuration, wherein in the closed configuration the resilient actuator is in a relaxed conformation with a first angle between each of the at least two resilient arms and the central piston member when no external force is applied to the resilient compressible body, and the distal plug is engaged with the distal drainage hole; and in the open configuration an external force is applied to the resilient compressible body and the at least two resilient arms, wherein the resilient actuator is in a compressed conformation with a second angle between each of the at least two resilient arms and the central piston member, the second angle being smaller than the first angle, and the distal plug is retracted from engagement with the distal drainage hole.

The valve may further include a proximal attachment mechanism operable to connect to a vessel containing a fluid. In some examples, the resilient compressible body has a distal plug guide adjacent to the distal drainage hole, and the distal plug guide may be operable to guide and position the distal plug in the distal drainage hole. The distal plug may have a distally tapering shape. In some examples, the distal plug may have a conical shape and the distal plug guide may have a shape that is complementary to the conical shape of the distal plug. In some examples, the at least two resilient arms each include an upper portion connected to the central piston member, lower portion, and a flexible elbow where the upper portion and the lower portion meet. In some examples, the at least two resilient arms may be mechanically connected to the interior surface of the compressible body. In some examples, the resilient actuator may include at least three resilient arms, wherein each of the at least three resilient arms is in mechanical contact with an interior surface of the resilient compressible body. In some examples, the surface of the distal plug includes a hydrophobic material or a hydrophilic material. In some examples, the interior surface of the resilient compressible body may be coated with a non-reactive material. In some examples, the resilient actuator may be coated with a non-reactive material.

In another aspect, the present invention may be directed to a method of manufacturing a valve comprising inserting an actuator into a resilient compressible body having a distal drainage hole, said actuator comprising a central piston member; at least two resilient arms, wherein each of said at least two resilient arms are connected to said central piston member; and a distal plug mechanically connected to said central piston member and operable to be inserted into and retracted from said distal drainage hole. The valve may have a closed configuration and an open configuration, wherein in the closed configuration the resilient actuator is in a relaxed conformation with a first angle between the at least two resilient arms when no external force is applied to the resilient compressible body, and the distal plug is engaged with the distal drainage hole; and in the open configuration an external force is applied to the resilient compressible body and the at least two resilient arms, wherein the resilient actuator is in a compressed conformation with a second angle between the at least two resilient arms, the second angle being smaller than the first angle, and the distal plug is retracted from engagement with the distal drainage hole.

In some examples, the valve may further comprising a proximal attachment mechanism operable to connect to a fluid conduit or reservoir. In some examples, the valve may further include a distal plug guide adjacent to the distal drainage hole operable to guide and position the distal plug in the distal drainage hole. The distal plug may have a distally tapering shape. The distal plug guide may have a shape that is complementary to the distally tapering shape of the distal plug. The resilient actuator may be coated with a non-reactive material. The interior of the resilient compressible body may be made from or coated with a non-reactive material. In some examples, the distal end of at least one of the at least two resilient arms may be in mechanical connection with an interior surface of the compressible body. In some examples, the at least two resilient arms includes an interfacing plate at a distal end thereof, the interfacing plate being in mechanical connection with the connection member. In some examples, actuator may further comprise a ring structure in mechanical connection with the at least two resilient arms and in mechanical contact with an interior of the compressible body. In some examples, the surface of the distal plug includes a hydrophobic material or a hydrophilic material. In some examples, the interior surface of the resilient compressible body may be coated with a non-reactive material. In some examples, the resilient actuator may be coated with a non-reactive material.

In another aspect, the present invention may be directed to a method of manufacturing a check valve comprising inserting an actuator into a tubing for passing a gas or fluid, said actuator comprising at least two arms mechanically connected to an interior of said tubing; a central piston member mechanically connected to said at least two arms, wherein each of said at least two arms is attached to the central piston member at an oblique angle with respect to the longitudinal axis of the central piston member; and a distal plug mechanically connected to a distal end of said central piston member; and wherein said tubing includes a plug receiver for receiving said distal plug, wherein said distal plug being operable to be inserted into and retracted from said plug receiver. The valve may have a closed configuration and an open configuration, wherein in the closed configuration the resilient actuator is in a relaxed conformation with the distal plug engaged with the plug receiver; and in the open configuration the resilient actuator is in a compressed conformation with the distal plug is retracted from engagement with the plug receiver. The actuator may have a pre-determined threshold resistance, such that when a gas or liquid applies a pressure equal to the pre-determined threshold resistance, the distal plug is unseated from the plug receiver and the check valve is opened.

In some examples, the distal plug may have a distally tapering shape. In some examples, the distal plug may have a conical shape and the distal plug guide may have a shape that is complementary to the conical shape. The at least two resilient arms may be mechanically connected to the interior surface of the tubing. The at least two arms may be mechanically connected to the interior surface of the compressible body. In some examples, the actuator may include at least three arms, wherein each of the at least three arms is in mechanical contact with an interior surface of the tubing. In some examples, the actuator may be coated with a hydrophobic material or a hydrophilic material. In some examples, the actuator may be coated with a non-reactive material.

It is an objective of the present invention to provide a durable innovative pinch valve for easily dispersing liquids, gases, and other flowable materials.

It is a further objective of the present invention to provide an innovative pinch valve that is easily and comfortably operated by the user without leakage or spillage.

It is a further objective of the present invention to provide an innovative pinch valve that allows a user to easily operate the pinch valve with one hand.

It is a further objective of the present invention to provide an innovative pinch valve that can be easily, conveniently, and cleanly used for draining waste receptacles (e.g., colostomy bags, urine receptacles, etc.) without spillage or leakage.

It is a further objective of the present invention to provide an innovative pinch valve having an internal, resilient actuation structure that, upon compression of the valve structure by the user, can retract a plug from a distal drainage hole and then reseat the plug in the distal drainage hole when the user releases the valve structure.

It is a further objective of the present invention to provide an innovative pinch valve may include non-reactive interior surface so it may disperse reactive (e.g., corrosive) chemicals without deterioration of the valve structure.

It is to be understood that both the general description and the following detailed description are exemplary and explanatory only and are not meant to limit the present invention. One with ordinary skill in the art will recognize that the general description and the detailed description will recognize that alterations and equivalents not described herein are captured in the spirit of the invention.

### BRIEF DESCRIPTION OF THE DRAWINGS

FIG. 1A shows a front view of a compressible valve according to some embodiments of the present invention.

FIG. 1B shows a cross sectional view of a compressible valve according to some embodiments of the present invention.

FIG. 1C show a cross sectional view of a compressible valve according to some embodiments of the present invention.

FIG. 2A shows a perspective view of a compressible valve according to some embodiments of the present invention.

FIG. 2B shows a perspective view of a compressible valve according to some embodiments of the present invention.

FIG. 3A shows a cross sectional view of a compressible valve according to some embodiments of the present invention.

FIG. 3B shows a cross sectional view of a compressible valve according to some embodiments of the present invention.

FIG. 3C shows a cross sectional view of a compressible valve according to some embodiments of the present invention.

FIG. 4A shows a perspective view of a compressible valve according to some embodiments of the present invention.

FIG. 4B shows a perspective view of a compressible valve according to some embodiments of the present invention.

FIG. 4C shows a cross sectional view of a compressible valve according to some embodiments of the present invention.

FIG. 5A shows a front view of a compressible valve according to some embodiments of the present invention.

FIG. 5B shows a cross sectional view of a compressible valve according to some embodiments of the present invention.

FIG. 6A shows a cross sectional view of a one-way check valve according to some embodiments of the present invention.

FIG. 6B shows a cross sectional view of a one-way check valve according to some embodiments of the present invention.

FIG. 6C shows a cross sectional view of a one-way check valve according to some embodiments of the present invention.

### DETAILED DESCRIPTION OF THE EXEMPLARY EMBODIMENTS

Reference will now be made in detail to certain embodiments of the invention, examples of which are illustrated in the accompanying drawings. While the invention will be described in reference to these embodiments, it will be understood that they are not intended to limit the invention. To the contrary, the invention is intended to cover alternatives, modifications, and equivalents that are included within the spirit and scope of the invention as defined by the claims. In the following disclosure, specific details are given to provide a thorough understanding of the invention. However, it will be apparent to one skilled in the art that the present invention may be practiced without these specific details.

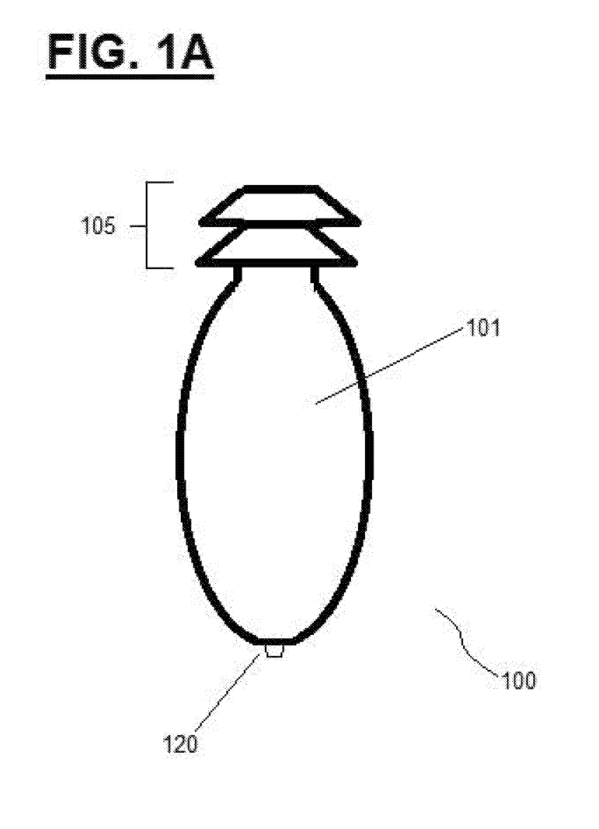

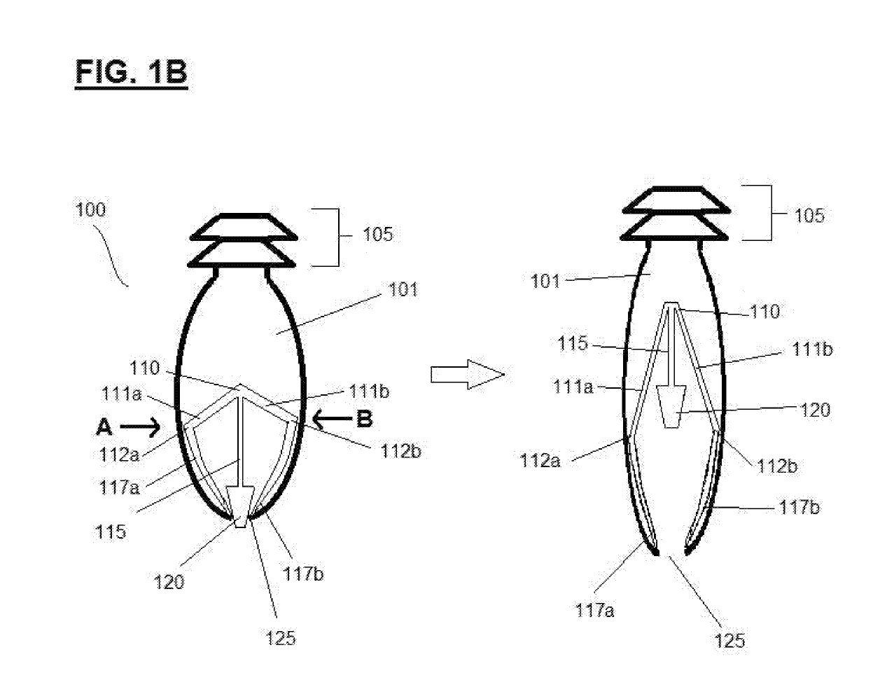

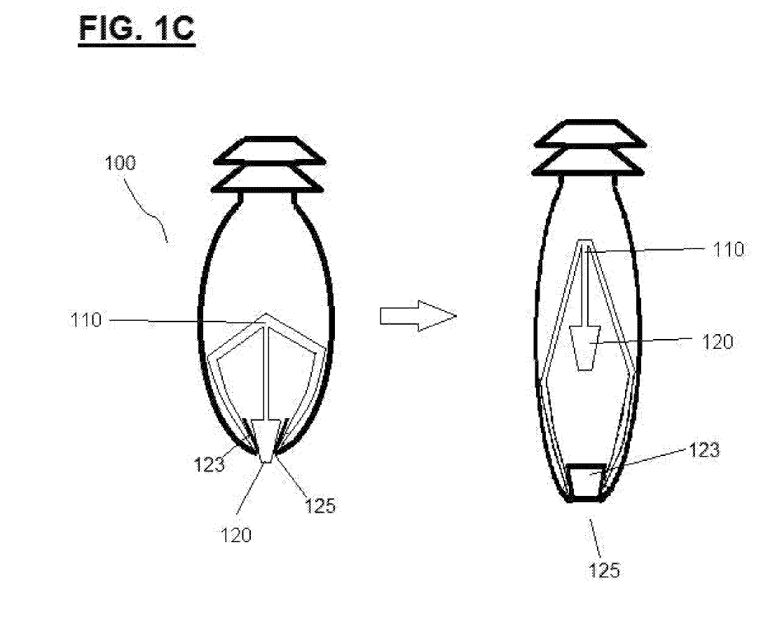

FIGS. 1A-1C illustrate exemplary embodiments of a one-way compressible pinch valve that may be used to dispense a liquid, gas, or other flowable material. FIGS. 1A-1B show a compressible valve 100 that includes a resilient compressible body 101, a proximal attachment mechanism 105, and a resilient actuator 110 nested within the resilient compressible body 101. The actuator 110 includes a central piston member 115, a distal plug 120, and two two resilient flexible arms 111a and 111b that radiate out from the central piston member 115 and contact the interior surface of the compressible body 101. FIG. 1B shows the resilient arms 111a and 111b may each include flexible elbows (112a and 112b) and lower arms (117a and 117b). The two resilient arms 111a and 111b may be in mechanical contact with the interior surface of the resilient compressible body 101. As shown in FIG. 1B, the flexible elbows 112a and 112b and a portion of the lower arms 117a and 117b may be in contact with the interior surface of the resilient compressible body 101. In some embodiments, the flexible elbows 112a and 112b may be mechanically connected (attached) to the interior surface of the compressible body 101 to aid in keeping the resilient actuator 110 in position within the resilient compressible body 101.

The resilient actuator 110 may be a durable, spring-like structure that compresses when the user squeezes the exterior of the compressible body and then resiles to its original shape once the user releases the compressible body 101. The central piston member 115 may be a rigid bar-like structure that is positioned along the longitudinal axis of the pinch valve 100. The proximal ends of the two resilient arms 111a and 111b may be attached to the proximal end of the central piston member 115. The distal end of the central piston member 115 may be rigidly and/or integrally connected to a distal plug 120 such that the central piston member 115 and the distal plug 120 are or are about coaxial. The angle between the central piston member 115 and the resilient arms 111a and 111b, respectively may be in a range of about 45° to about 80°. The distal ends of the two or more arms may contact the inner wall of the compressible body at compression points A and B at which the user applies force to the outer side of the compressible body 101. As the user applies compressive force, the two resilient arms are pressed inward toward the central piston member, and the joint angle between each resilient arm and the central piston member decreases (e.g., without limitation, by an amount of from about 20° to about 70°) and the central piston member moves proximally as the joint angles decrease. As the central piston member moves proximally, the distal plug 120 may be disengaged from the distal drainage hole 125. The actuator may maintain its position within the resilient compressible body 101 and thereby allow the distal plug 120 to return to its original position in the distal drainage hole 125 by resiling to its original shape.

The distal plug 120 may have a shape that is operable to obstruct a distal drainage hole 125 at the distal end of the resilient compressible body 101, thereby preventing any fluid, gas, or other flowable material from escaping through the distal drainage hole 125 when the valve is in the closed condition. The distal plug 120 may have a distally tapering shape (e.g., a conical shape) so that it may best create a seal when engaged with the distal drainage hole 125 of the compressible body 101. It is to be understood that the distal plug may various distally tapering shapes (e.g., a sphere, an ellipsoid, a pyramidal wedge structure, etc.) and that the distal drainage hole may have a shape that is complementary to the shape of the distal drainage plug.

The proximal attachment mechanism 105 may allow the valve 100 to be detachable from a liquid or gas source (e.g., reservoir, container, tank, etc.). As shown in FIG. 1A, the proximal attachment mechanism 105 may be a conduit structure having circumferential barbs or ridges for frictionally engaging an interior of a drainage conduit on the liquid or gas source. The ridges may serve to pressure fit and/or engage with complementary ridges within the drainage conduit on the liquid or gas source. In other implementations, the proximal attachment mechanism may have a different connection mechanism, such as threading or a quick-connect device. In still other implementations, the proximal attachment mechanism may be continuously and integrally formed with the liquid or gas source.

The interior of the resilient compressible body 101 of FIGS. 1A-B may be made from or coated with a non-reactive material (e.g., polyurethane, PVC, PTFE perfluoroelastomers (FFKM), etc.) which allows the valve 101 to contain corrosive liquids (e.g., acids, bases, etc.), vapors, gases (e.g., chlorine, ammonia, etc.). The non-reactive coating of the interior of the resilient compressible body 101 may help with the longevity of the life of the valve by protecting the interior of the resilient compressible body 101 from corrosion.

FIG. 1B shows the valve in both the closed and open configurations. The function of the actuator 110 is to allow for the release of the liquid, gas, or other flowable material from the source to which the valve 100 is connected (e.g., a reservoir, container, tank, etc.). When in the closed configuration the distal plug 120 is seated in the distal drainage hole 125 and the liquid, gas, or other flowable material is contained within the valve 100 and cannot flow through the distal drainage hole 125. Upon pinching at or near compression points A and B of the resilient compressible body 101, the two flexible elbows 112a and 112b are squeezed and the resilient arms 111a and 111b are elongated within the compressible body 101. As the elbows 112a and 112b are squeezed and elongated, the angle between the upper and lower portions of each resilient arm increases, and the angles between the resilient arms 111a and 111b and the central piston member 115 decrease. As a result, the central piston member 115 and the distal plug 120 are proximally retracted, thereby opening the distal drainage hole 125. In this open configuration, the distal plug 120 of the valve 100 is removed from the distal drainage hole 125, thereby allowing the liquid, gas, or other flowable material to be released from the source (reservoir, container, tank, etc.) through the valve 100.

When the user releases the compressible body 101 of the valve 100, the resilient actuator 110 relaxes and resiles to its original shape, thereby moving the central piston member 115 distally and reseating the distal plug 120 in the distal drainage hole 125. The simple manual squeezing actuation of the valve 100 and the bias of the resilient actuator toward the closed position allows for very easy and efficient operation by the user. The valves of the present invention are easily opened by applying pressure to the compression points (which may be marked by button like protrusion, color markings, and/or other distinguishing mechanisms) and automatically closed by simply releasing the compressible body 101. The valve 100 can be easily and efficiently opened, unlike the clumsy mechanisms found in conventional fluid valve designs.

FIG. 1C illustrates an embodiment of a valve according to the present invention that additionally includes a distal plug guide 123 adjacent to the distal drainage hole 125. The distal plug guide 123 may be operable to aid in guiding 123 the distal plug 120 into alignment with the distal drainage hole 125. The distal plug guide may assist in preventing the distal plug 120 from becoming misaligned from the distal drainage hole 125 when lowering the distal plug 120 back towards the distal drainage hole 125. The distal plug guide 123 may have a complimentary shape to the distal plug 120 providing addition interface surface area with the distal plug 120, aiding in creating a tight seal at the distal drainage hole. The distal plug guide 123 may be a solid frustrum shape concentric with the distal drainage hole 125 as shown in FIG. 1C. In other implementations, the distal plug guide may be made up of two or more angled plates that approximate the frustrum of a cone, and function in the same manner as the frustrum-shaped distal plug guide 123. The distal plug guide 123 show in FIG. 1C may be made of or coated with a non-reactive material (e.g., polyurethane, PVC, PTFE perfluoroelastomers (FFKM), etc.). In another embodiment, the distal plug guide 123 may be coated with a hydrophobic (e.g., where the valve is retaining polar liquids) or hydrophilic layer (e.g., where the valve is retaining nonpolar liquids) to aid in preventing leakage of the material therein.

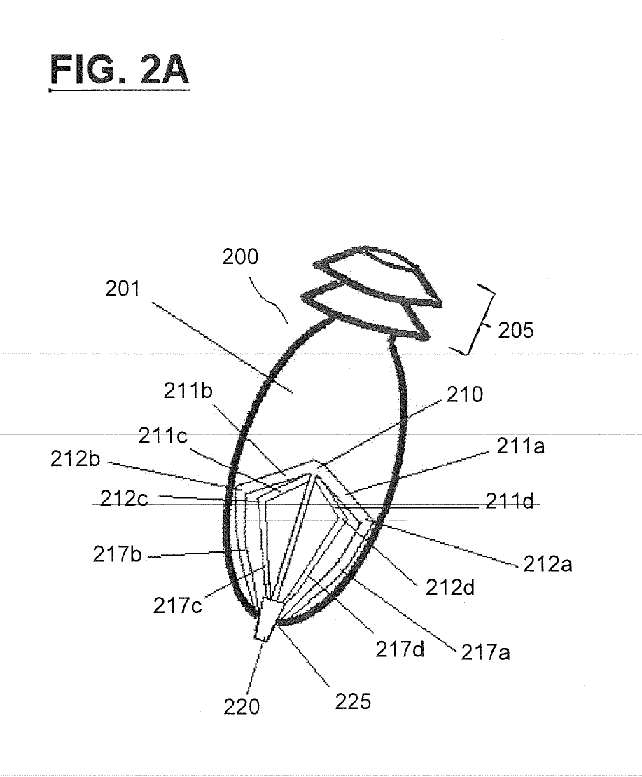

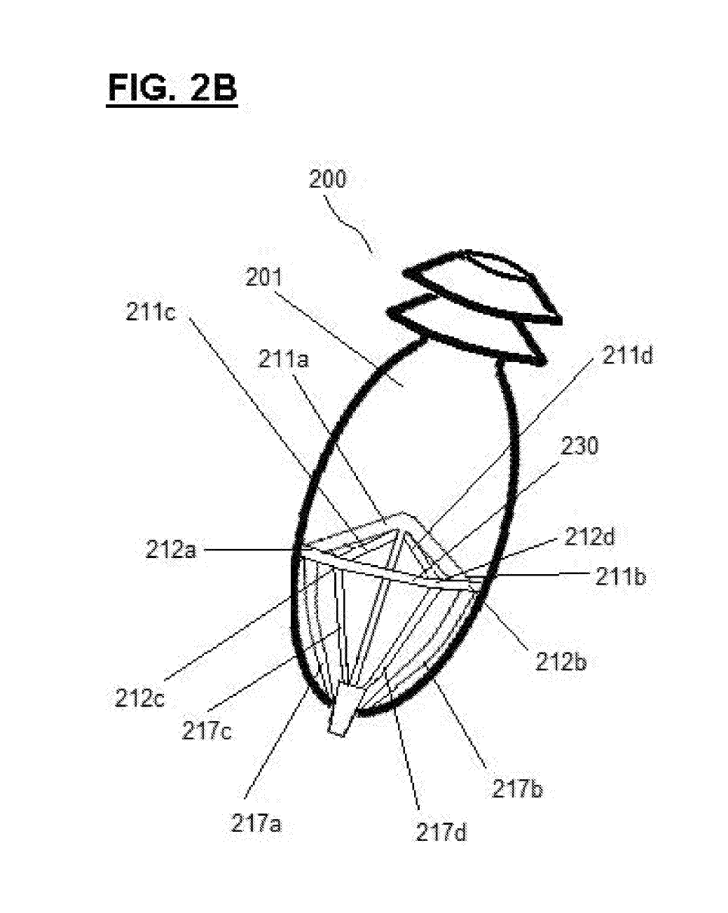

FIG. 2A shows another exemplary embodiment of a pinch valve according to the present invention. Aside from the actuator 210, the structures of the pinch valve 200 shown in FIG. 2A are the same as or similar to the corresponding structures in FIGS. 1A-1B. The pinch valve includes a compressible body 201, a proximal attachment mechanism 205, and an distal drainage hole 225. The resilient actuator 210 differs somewhat in design from the embodiment shown in FIGS. 1A-1C. The resilient actuator 210 includes a central piston member 215 and a distal plug 220 that may be seated in distal drainage hole 225. The resilient actuator additionally includes four resilient arms 211a, 211b, 211c, and 211d, each having a flexible elbow (212a, 212b, 212c, and 212d, respectively) and a resilient lower arm (217a, 217b, 217c, and 217d, respectively). The four resilient arms may be radially spaced from each other in a symmetrical or about symmetrical arrangement such that there is about a 90° between each pair of adjacent resilient arms (e.g., the radial angle between arm 211a and 211b may be about 90°). However, the four resilient arms may be spaced in other arrangements. For example, each arm may be spaced at first radial angle with respect to an adjacent arm on one side thereof (e.g., 45°) and a second radial angle with respect to an adjacent arm on the other side thereof (e.g., 135′). The presence of four radiating resilient arms in this embodiment increases the compression points at which the user can apply pressure to squeeze and compress the actuator. The additional points at which the actuator 210 may be squeezed allows the user to more easily handle and open the pinch valve 200.

It is to be understood that while FIG. 2A shows four resilient arms, flexible elbows, and lower arms, further embodiments of the valve may include additional arms (e.g., 6, 8, 10, etc.). With additional arms, the pinch valve may be able to be compressed between any two diametric or about diametric points on the circumference of the resilient compressible body and compress the resilient actuator. Upon engaging the flexible elbows by compressing the resilient compressible body, the flexible elbows are extended thereby decreasing the angle between the resilient arms and proximally retracting the central piston member and distal plug from the distal drainage hole thereby opening the valve.

FIG. 2B shows an embodiment of the valve 200 wherein the resilient actuator 210 includes a further ring structure 230 that connects with the elbows 212a, 212b, 212c, and 212d of resilient arms 211a, 211b, 211c, and 211d. The ring structure 230 may be in mechanical contact with the interior of the resilient compressible body 201 of the valve 200. The ring structure 230 may have a substantially complementary shape to the interior perimeter of the resilient compressible body 201 (e.g., a ring, polygon, etc.). The ring may be positioned adjacent to the interior perimeter of the compressible body on a plane that is or is about perpendicular to the longitudinal axis of the pinch valve 200. In some examples, the interior perimeter may include a recess, receiver, tabs, or other structure that engages with the ring structure to assist in holding it in place. Additionally, the at least two resilient lower arms 217a, 217b, 217c, and 217d may be in mechanical connection with the resilient compressible body 201 to increase the interfacing area of the resilient actuator 210 within the resilient compressible body 201. The presence of ring structure in this embodiment increases the compression points at which the user can apply pressure to squeeze and compress the actuator, and may allow the user to squeeze the compressible body at any point on the exterior perimeter thereof near the ring structure. It is to be understood that additional ring structure(s) or other structures that increase the interfacing area between the interior of the compressible body and the actuator may be present. For example, the actuator may include an additional ring structure adjacent to the first ring structure, attached to the lower arms. In another example, plate or disk-like structures (e.g., circular, polygonal, or other shapes) may be attached to the elbows of the resilient arms to increase the interfacing area of the actuator and the compressible body to allow the user broader compression points for operating the valve.

Variations in the valve features depicted in FIGS. 2A-2B are within the scope of the present invention. One with ordinary skill in the art will appreciate that the number of resilient arms, presence or absence of a distal plug guide, and presence or absence of a ring structure all fall within the scope of the present invention. Various combinations and permutations of the features described above are all within the spirit of the invention.

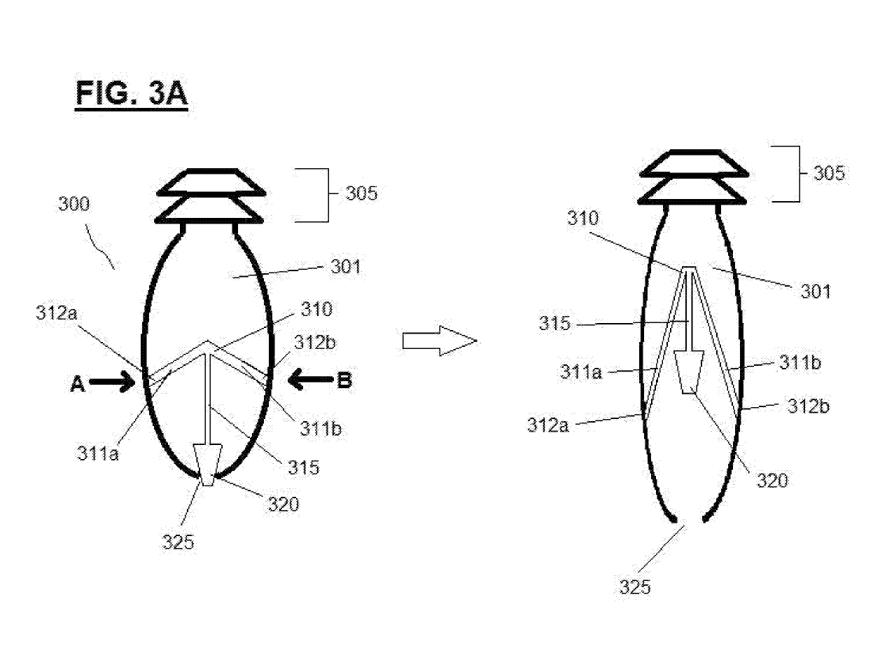

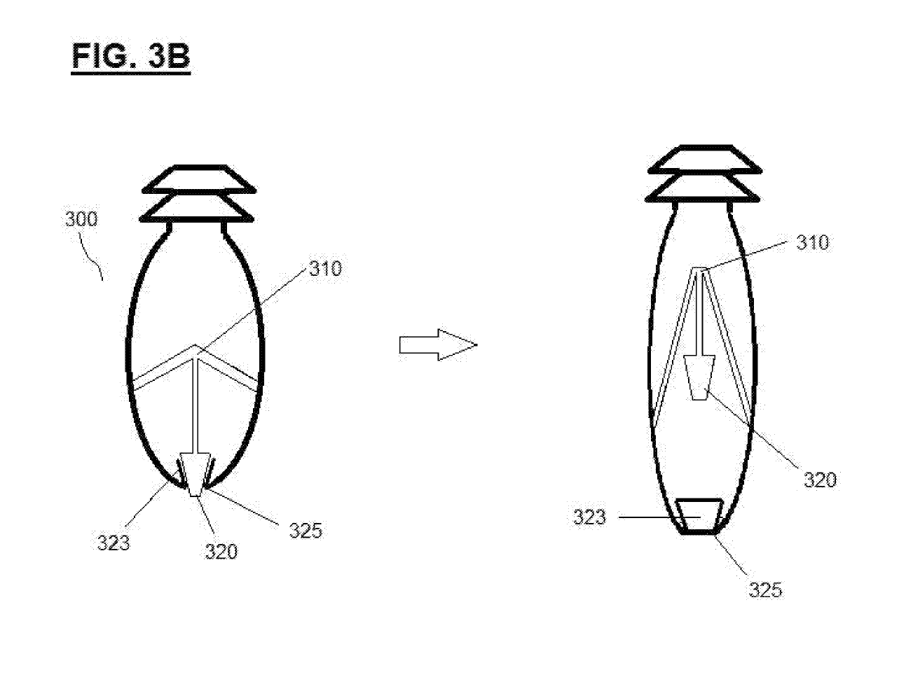

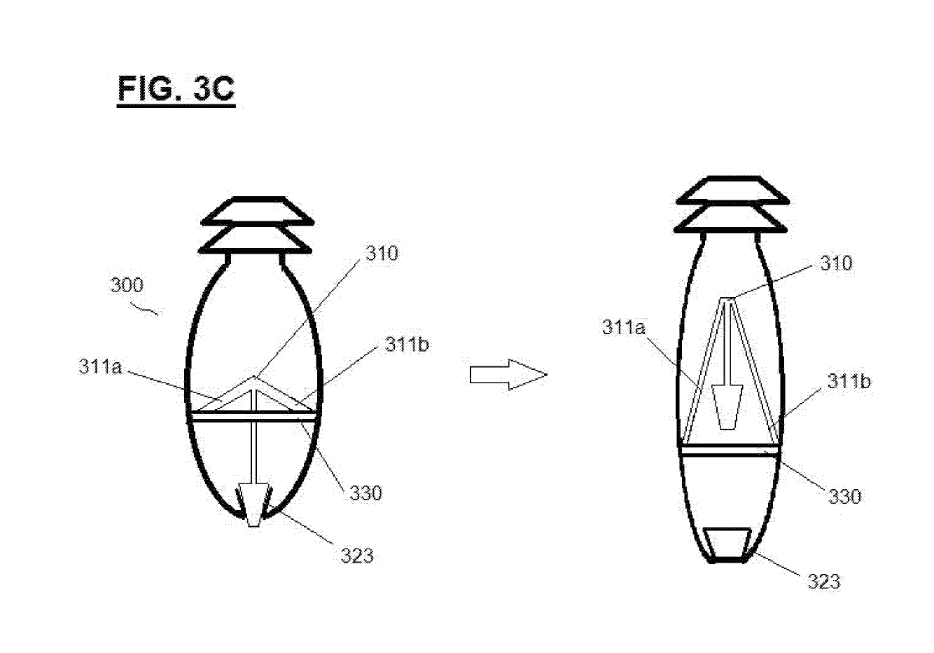

FIGS. 3A-3C show alternate embodiments of the pinch valve. FIG. 3A shows the valve 300 has a resilient compressible body 301, a proximal attachment mechanism 305, a resilient actuator 310 that includes two resilient flexible arms 311a and 311b that project distally within the resilient compressible body 301. The compressible body 301 is the same or substantially similar to the compressible body as described with regard to FIGS. 1A-1C and 2A-2B. FIG. 3A shows the two resilient arms 311a and 311b each have distal connection points 312a and 312b which may be mechanically connected to the interior of the compressible body 301 such that the resilient actuator 310 is kept in position within the compressible body 301. The resilient actuator 310 may also have a central piston member 315 connected to a distal plug 320. The distal plug 320 may have a distally tapering shape that is operable to block a distal drainage hole 325 at the distal end of the resilient compressible body 301, thereby preventing any liquid, gas, or other flowable material from escaping through the distal drainage hole 325.

FIG. 3A shows the valve 300 in both closed and open configurations. In the closed position the distal plug 320 is seated in the distal drainage hole 325. When a user squeezes the pinch valve 300 at compression points A and B, the distal connection points 312a and 312b are compressed, and the angle between resilient arms 311a and 311b the central piston member 310 decrease, thereby proximally retracting the central piston member 315 and the distal plug 320 and placing the valve 300 in the open configuration.

The resilient actuator 310 is a durable spring-like structure that compresses when the user squeezes the exterior of the compressible body 301 and then resiles to its original shape when the user releases the compressible body 301. The central piston member 315 may be a rigid bar-like structure that is positioned along the longitudinal axis of the pinch valve 300. The proximal ends of the two resilient arms 311a and 311b may be attached to the proximal end of the central piston member 315. The distal end of the central piston member 315 may be rigidly and/or integrally connected to a distal plug 320 such that the central piston member 315 and the distal plug 320 are or are about coaxial. The angle between the central piston member 315 and the resilient arms 311a and 311b, respectively, may be in a range of about 45° to about 80°. The distal ends of the two or more arms contact the inner wall of the compressible body at compression points A and B at which the user applies force to the outer side of the compressible body 301. As the user applies compressive force, the two resilient arms are pressed inward toward the central piston member, and the joint angle between each resilient arm and the central piston member decreases (e.g., without limitation, by an amount of from about 20° to about 70°) and the central piston member moves proximally as the joint angles decrease. As the central piston member moves proximally, the distal plug may be disengaged from the distal drainage hole 325. The actuator may maintain its position within the resilient compressible body 301 and thereby allow the distal plug 320 to return to its original position by resiling to its original shape.

FIG. 3B illustrates an embodiment of the valve 300 that includes a distal plug guide 323 adjacent to the distal drainage hole 325. The resilient actuator 310 functions in a similar manner as described in FIG. 3A. The distal plug guide 323 aids in aligning the distal plug 320 in the distal drainage hole 325 should the distal plug 320 become misaligned with the distal drainage hole 325. Additionally, the distal plug guide 323 may have a complimentary shape to the distally tapering distal plug 320 to aid in sealing in the liquid, gas, or other flowable material and prevent leakage. For example, the distal plug 320 may have a conical shape and distal plug 323 may shaped like the frustrum of a cone. In other implementations, the distal plug guide may be made up of two or more angled plates that approximate a shape that is complementary to the distal plug (e.g., the frustrum of a cone).

FIG. 3C depicts an embodiment of the compressible valve 300 which the resilient arms 311a and 311b may be connected to a ring structure 330. Ring structure 330 may be the same as or similar to the ring structure discussed above with regard to FIG. 2B. The ring structure 330 may be in mechanical contact with the interior of the compressible body 301. The ring structure 330 may have a substantially complementary shape to the interior perimeter of the resilient compressible body 301 (e.g., a ring, polygon, etc.). The ring may be positioned adjacent to the interior perimeter of the compressible body on a plane that is perpendicular to the longitudinal axis of the pinch valve. In some examples, the interior perimeter may include a recess, receiver, tabs, or other structure that engages with the ring structure to assist in holding it in place. As the user squeezes the compressible body 301, the resilient arms 311a and 311b connected to the ring structure 330 are compressed, which, in turn, reduces the angle between the resilient arms and the central piston member. As a result, the distal plug 320 is proximally retracted from the distal drainage hole 325, thereby opening the valve 300.

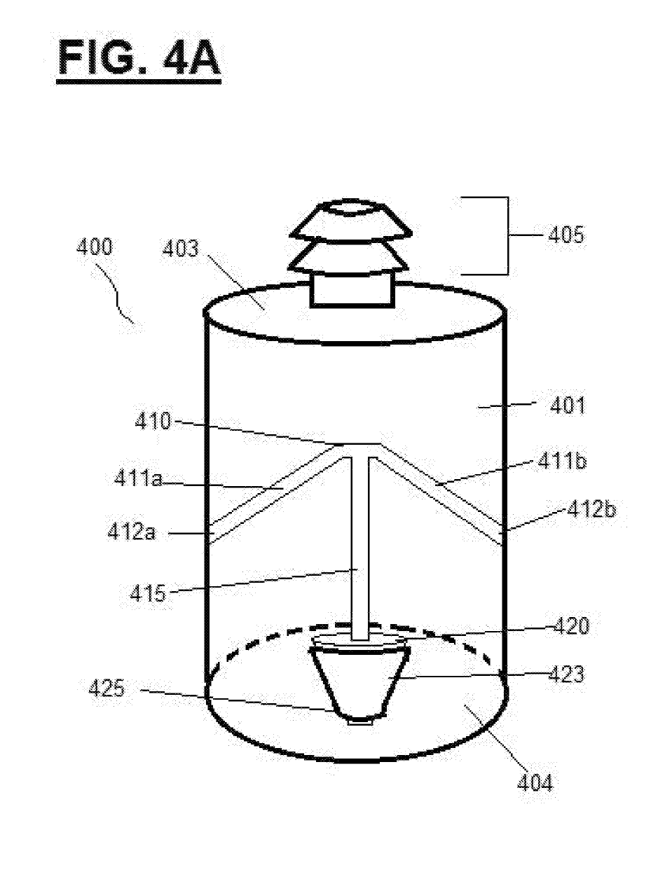

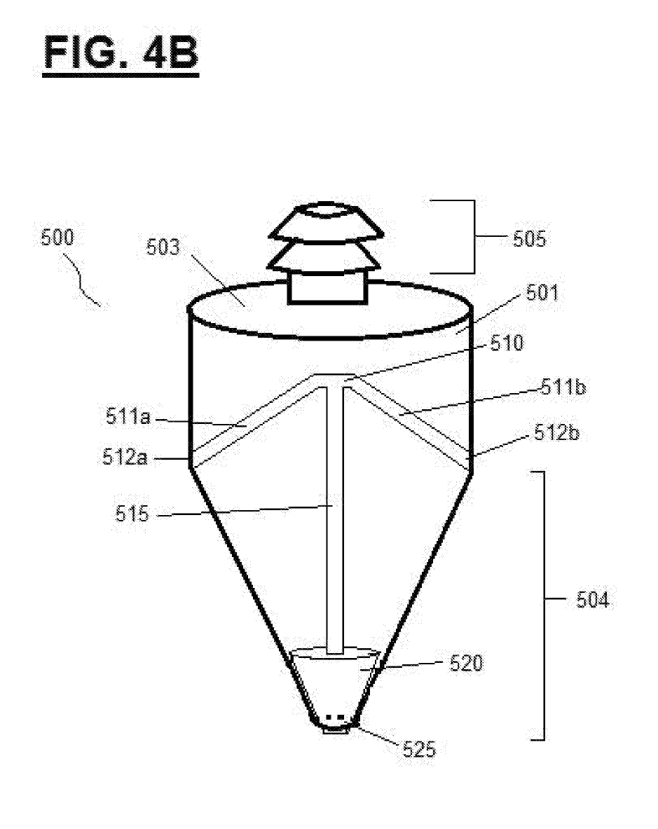

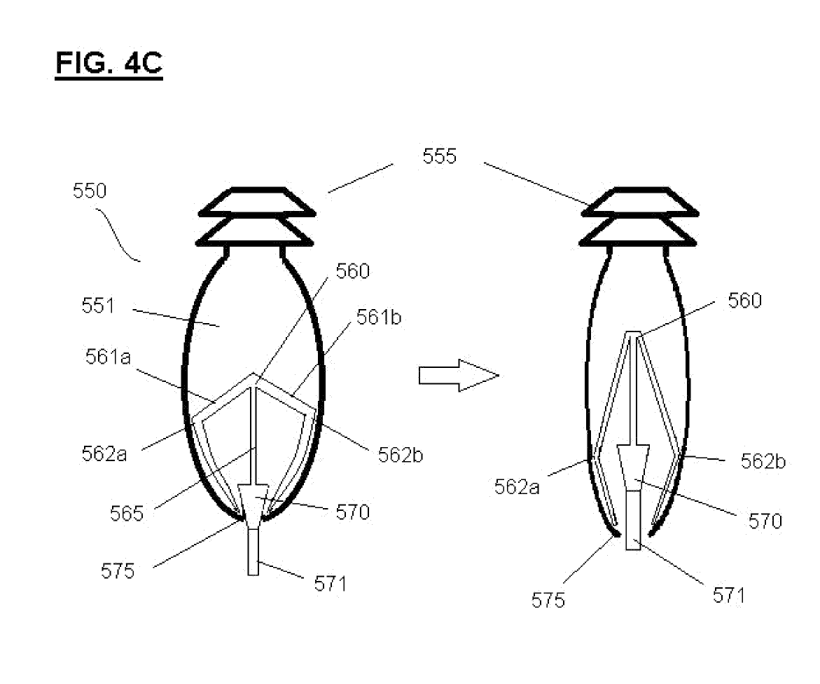

FIGS. 4A-4B show a compressible valve with exemplary alternative shapes for the compressible body. It is to be appreciated that the following illustrations are not meant to be limiting or exhaustive; one with ordinary skill in the art will appreciate that the resilient compressible body can take many other shapes.

FIG. 4A depicts a pinch valve 400 with a cylindrically shaped resilient compressible body 401. The valve 400 may have a proximal end 403, a distal end 404, a proximal attachment mechanism 405, a distal plug 420, a distal plug guide 423, and a distal drainage hole 425. The proximal attachment mechanism is operable to connect (and disconnect) the compressible valve 400 to a source containing a liquid, gas, or other flowable material (e.g., reservoir, container, tank, etc.). A resilient actuator 410 may be positioned within the compressible body 401, and may be substantially similar in structure and function to actuators described herein with respect to FIGS. 3A-3C. As in other embodiments, the resilient actuator 410 is a durable, spring-like structure that resiles to its original shape once the resilient compressible body 401 is no longer compressed, thereby returning the distal plug 420 into original position to obstruct the distal drainage hole 425.

As in other embodiments described herein, the distal plug guide 423 may aid in aligning the distal plug 420 with the distal drainage hole 425. Additionally, the distal plug guide 423 may have a complimentary shape to the distal plug 420 to aid in sealing the valve 400 and prevent leakage of the contained liquid, gas, or other flowable material by providing more interfacing surface area with the distal plug 420. In other implementations, the distal plug guide may be made up of two or more angled plates that approximate a complementary shape to the distal plug.

FIG. 4B illustrates an exemplary embodiment of a pinch valve 500 having a resilient compressible body 501 having a distally tapering shape. The pinch valve 500 has a proximal end 503, a conical portion 504, a proximal attachment mechanism 505, and a distal drainage hole 525. The proximal attachment mechanism is operable to connect (and disconnect) the pinch valve 500 to a source of a liquid, gas, or flowable material (e.g., reservoir, container, tank, etc.). The pinch valve 500 may also include a resilient actuator 510, which may the same as or similar to the resilient actuators as described herein with regard to FIGS. 3A-3C. The resilient actuator 510 may include radiating arms 511a and 511b having distal ends 512a and 512b that are in mechanical contact with the interior surface of the compressible body 501 at compression points. The resilient actuator 510 is a durable spring-like structure that resiles to its original shape after being squeezed by the user, thereby returning the distal plug 520 into original position to block the distal drainage hole 525. In some implementations, the actuator may maintain its position within the resilient compressible body 501 by mechanical connection between the distal ends 512a and 512b of the resilient arms 511a and 511b and interior wall of the compressible body 502. In the present embodiment, the conical portion 504 of the compressible body 501 may serve as a distal plug guide to aid in aligning the distal plug 520 into alignment with the distal drainage hole 525.

In some embodiments, and without limitation, the distal plug may include a distal guide pin that passes through the distal drainage hole, and has a sufficient length such that the distal guide pin cannot be fully retracted into the body of the valve and at least a portion always remains on the exterior side of the distal drainage hole. Such a distal guide pin may aid in keeping the distal plug aligned with the distal drainage hole. FIG. 4C illustrates an exemplary embodiment of a pinch valve 550 having that includes a distal plug 570 having a distal guide pin 571 attached to a distal end thereof. As shown FIG. 4C, a portion of the distal guide pin 571 traverses the distal drainage hole 575 when the actuator 560 is compressed and the distal plug 570 is retracted from the distal drainage hole 575. The distal guide pin 571 aids in preventing the distal plug 570 from becoming misaligned with respect to the distal drainage hole 575. The pinch valve 550 may otherwise include similar features to those of other embodiments described herein. The pinch valve 550 may include a resilient compressible body 551 having a proximal attachment mechanism 555 and a distal drainage hole 575. The resilient actuator 560 may have the same as or similar to the resilient actuators as described herein with regard to FIGS. 1B-1C, or similar to other embodiments described herein. The resilient actuator 560 may include radiating arms 561a and 561b having elbows 562a and 562b that are in mechanical contact with the interior surface of the compressible body 551 at compression points. The resilient actuator 560 is a durable spring-like structure that resiles to its original shape after being squeezed by the user, thereby returning the distal plug 570 into original position to block the distal drainage hole 575. In some implementations, the actuator may maintain its position within the resilient compressible body 501 by mechanical connection between the elbows 562a and 562b of the resilient arms 561a and 561b and interior wall of the compressible body.

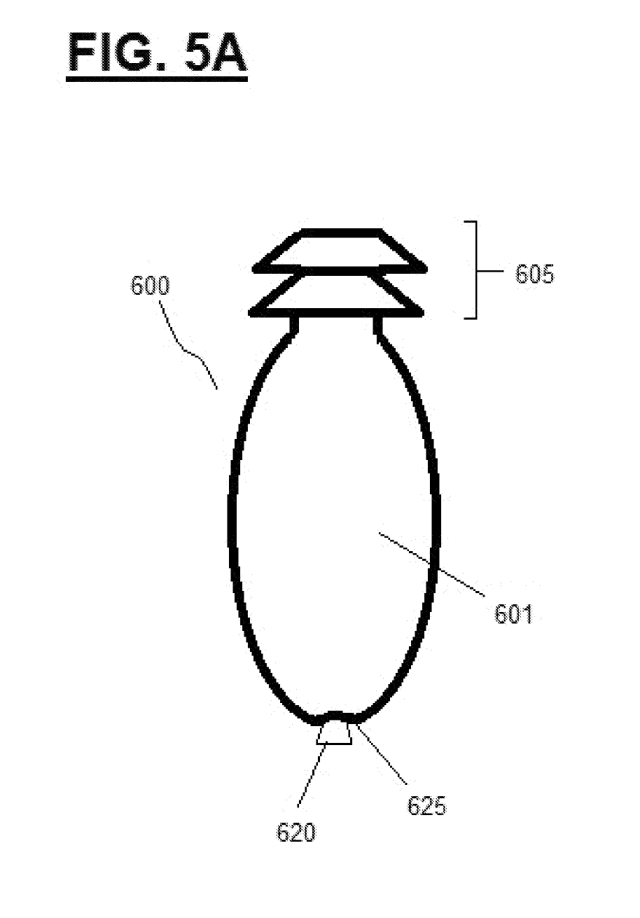

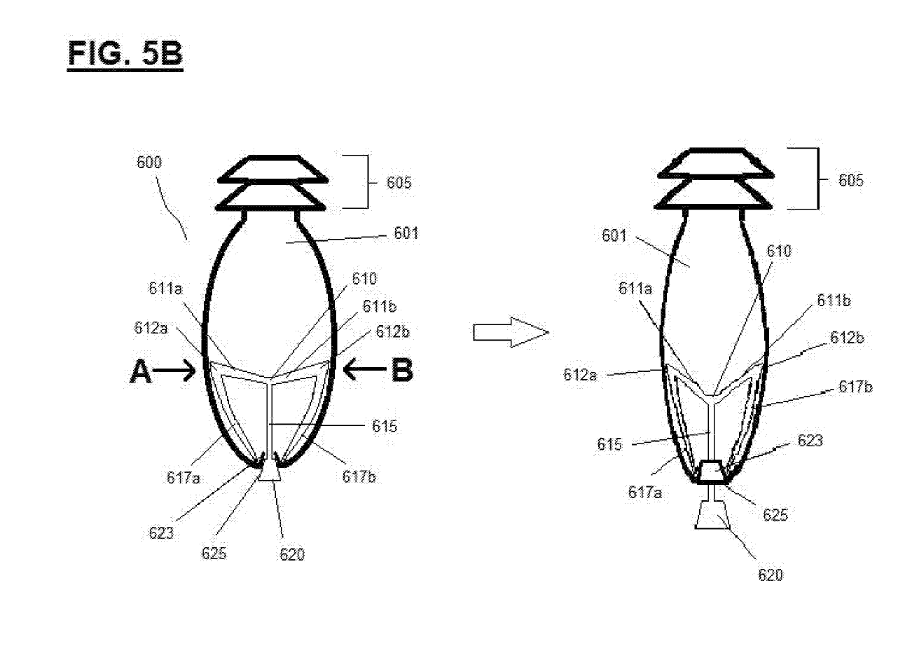

It is to be understood that the scope of the present invention includes other designs of the actuator. For example, the actuator may be configured such that the distal plug seals the compressible body from the exterior side of the distal drainage hole. FIGS. 5A-5B show an exemplary embodiment of the present invention in which the pinch valve 600 comprises an actuator 610 that includes a distal plug 620 that moves distally when the compressible body 601 is squeezed and that reseats by moving proximally when the user releases the compressible body 601.

The pinch valve 600 includes a resilient compressible body 601 with a proximal attachment mechanism 605, a resilient actuator 610 that includes two resilient flexible arms 611a and 611b that radiate from a central piston member 615. FIG. 5A provides a view of the exterior of the compressible body 601, which may be a resilient structure that can be compressed by the user and then return to its original shape once it is released. The proximal attachment mechanism 605 (e.g., rubber barbs, threading, Storz connector, etc.) may allow the valve 600 to be detachably to a source of liquid, gas, or other flowable material (e.g., reservoir, container, tank, etc.).

FIG. 5B provides a view of the interior of the compressible body 601, in which the resilient actuator 610 is depicted. The resilient actuator 610 includes a central piston member 615 connected to a distal plug 625. The distal plug 615 may have a proximally tapering shape that is operable to obstruct a distal drainage hole 625 at the distal end of the compressible body 601, thereby preventing liquid, gas, or other flowable material from escaping through the distal drainage hole 625. For example, the distal plug 620 may be shaped like a frustrum of a cone, where the narrow end of the frustrum is attached to the distal end of the central piston member 615. The frustum shape may create a seal when engaged with the distal drainage hole 625 of the compressible body 601. It is to be understood that the distal plug may have other proximally tapered shapes that are effective to seal the distal drainage hole (e.g., an ellipsoid or spherical shape).

The actuator 610 includes two resilient arms 611a and 611b that may each have flexible elbows 612a and 612b which may be mechanically contacting (or may be mechanically connected to) the interior of the compressible body 601. The resilient actuator 610 may also have lower arms 617a and 617b which may be mechanically contacting (or may be mechanically connected to) a distal portion of the interior wall of the compressible body 601. The actuator 610 may be held in position in the compressible body 601 by the contact (or connection) between the elbows 612a and 612b and the interior of the compressible body 601, and the contact (or connection) between the lower arms 617a and 617b and the interior of the compressible body 601.

FIG. 5B shows the pinch valve 600 in a closed configuration and an open configuration. A user may pinch the compressible body 601 at or near compression points A and B, which squeezes the flexible elbows 612a and 612b of the resilient compressible body 601 within the compressible body 601. As the elbows 612a and 612b are squeezed and compressed, the angle is increased between the central piston member 615 and the flexible arms 611a and 611b where the flexible arms meet the central piston member. Also, the angle between the upper and lower portions of the resilient arms 611a and 611b decrease. As a result, the central piston member 615 and distal plug 620 move distally and the distal plug 620 is unseated from the distal drainage hole 625 and the valve 600 is opened. The actuator 610 is a durable structure that resiles to its original shape once the users releases the compressible body 601, and the central piston member 615 and distal plug 620 are retracted proximally, reseating the distal plug 620 in the distal drainage hole 625.

The compressible body 601 may include a distal plug guide 623 having a complimentary shape to the distal plug 620. The distal plug guide 623 may increase the interfacing surface area between the distal plug 620 and the compressible body 601, and may aid to create a better seal between the distal plug 620 and the distal drainage hole 625.

In some embodiments, the present invention may be directed to a one-way, in-line check valve in a gas or fluid line. In such embodiments, the resilient actuator may be positioned within the gas or fluid line adjacent to a plug receiver for a distal plug of the actuator, such that the check valve is closed when the distal plug is seated in the plug receiver. The check valve may be opened when a the gas or fluid applies sufficient pressure (e.g., a predetermined threshold pressure, to which the actuator may be calibrated) to the distal end of the distal plug, thereby unseating the distal plug from the plug receiver and opening the one-way check valve. Once the pressure falls below the threshold pressure, the force of the compressed actuator reseats the distal plug in the plug receiver, thereby closing the one-way check valve.

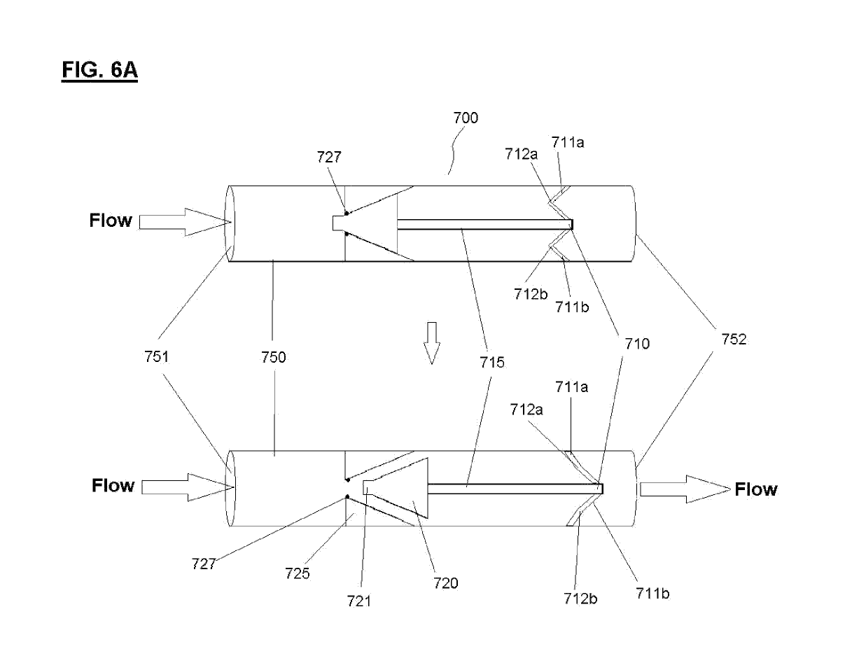

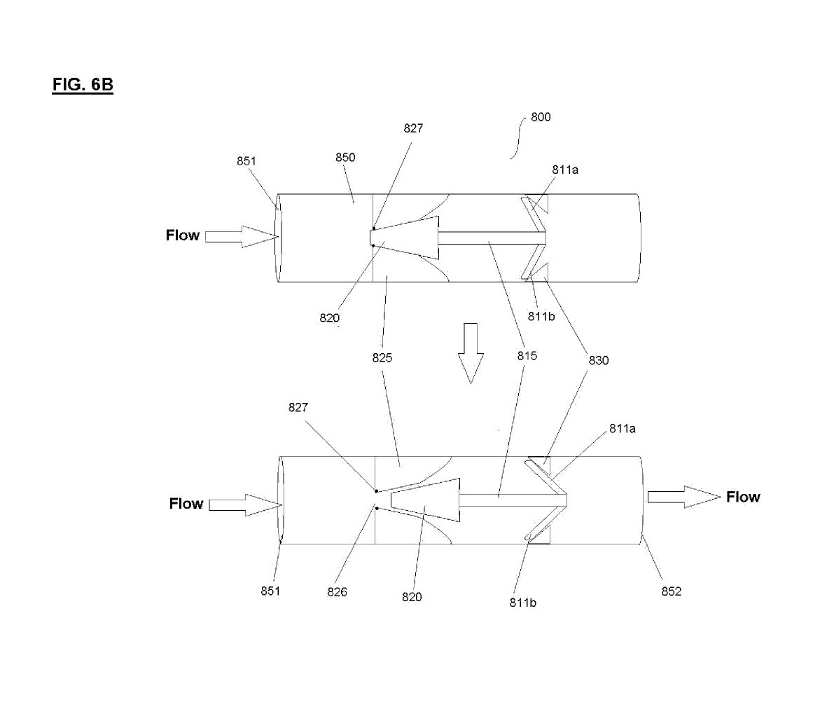

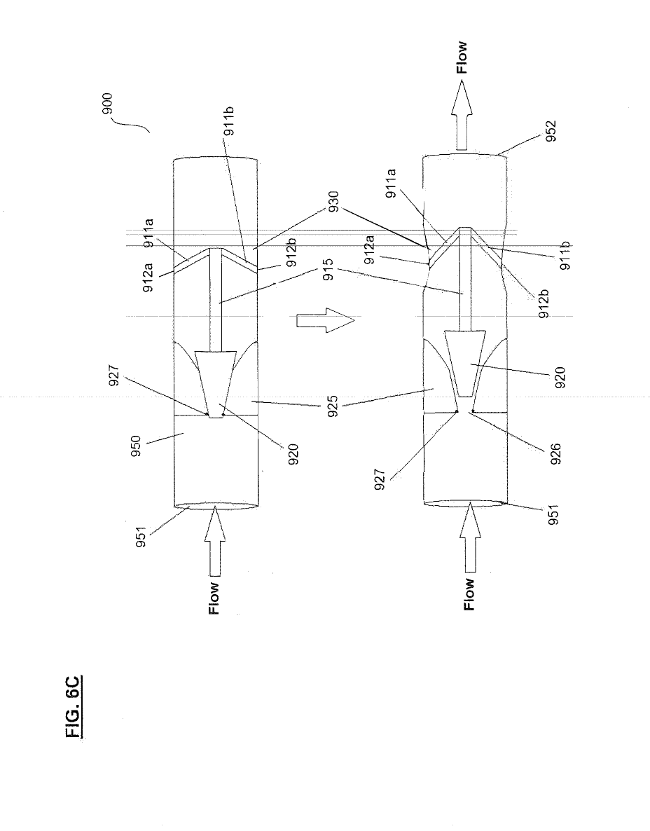

FIG. 6A shows an example of an in-line check valve 700 according to an embodiment of the present invention. The valve 700 may be positioned within tubing 750 for passing a gas, liquid, or other flowable material. The direction of flow is indicated by the arrows in the figure. The valve 700 includes an actuator 710 having a piston 715 and a distal plug 720 having a distally tapering shape (e.g., conical) and resilient flexible arms 711a and 711b. In the tubing 750, a plug receiver 725 may be formed therein having a complementary shape to the distal plug 720. The distally tapering shape of the distal plug 720 is operable to engage and obstruct the plug receiver 725, thereby preventing liquid, gas, or other flowable material from passing through the tubing 750. The distal plug receiver may include a gasket 727 (e.g., an o-ring) that may aid in sealing the valve 700 when the distal plug 720 is engaged with the distal plug receiver 725. The distal plug 720 may have a guide pin 721 help keep the distal plug aligned with the plug receiver 725 and help seal the valve 700. The resilient flexible arms 711a and 711b of the actuator 710 may have flexible elbows 712a and 712b than allow the arms to flex when the gas or fluid applies a threshold pressure to the distal end of the distal plug 720. Additionally, the attachment points of the resilient flexible arms 711a and 711b to the interior wall of the tubing 750 and the proximal end of the piston 715 may be flexible such that as the threshold pressure is applied to the distal end of the distal plug 720 and the distal plug 720 is unseated from the plug receiver 725, the angles between the resilient flexible arms and the interior wall of the tubing 750 may change and the angles between the resilient flexible arms and the piston 715 may change.

FIG. 6A provides successive views of the valve 700 during the process of the distal plug 720 being unseated from the plug receiver. The gas, fluid, or other flowable material flows through end 751 toward the distal plug 720, as shown in the top view (the closed condition). As the gas, fluid, or other flowable material reaches the pre-determined threshold pressure, it pushes on the distal end of the distal plug 720 (e.g., against guide pin 721) and unseats the distal plug 720 from the plug receiver 725, as shown in the bottom view (the open condition). In the open condition, the gas, fluid, or other flowable material flows through the valve 700 from the upstream side 751 to the downstream side 752 of the valve 700. Once the pressure of the gas, fluid, or other flowable material falls below the pre-determined threshold pressure, the spring-like action of the resilient actuator (i.e., the resilience of the resilient arms) causes the actuator to resile to its original shape with the distal plug 720 seated in the plug receiver 725 in the closed condition.

FIG. 6B shows an example of an in-line check valve 800 according to an embodiment of the present invention. The valve 800 may be positioned within tubing 850 for passing a gas, liquid, or other flowable material. The direction of flow is indicated by the arrows in the figure. The valve 800 includes an actuator 810 having a piston 815 and a distal plug 820 having a distally tapering shape (e.g., conical) and resilient flexible arms 811a and 811b. The resilient flexible arms 811a and 811b of the actuator 810 may be attached to the proximal end of the piston at flexible joints that allow the arms to flex toward the piston. In the tubing 850, a plug receiver 825 may be formed therein having a complementary shape to the distal plug 820. The distally tapering shape of the distal plug 820 is operable to engage and obstruct the plug receiver 825, thereby preventing liquid, gas, or other flowable material from passing through the tubing 850. The distal plug receiver may include a gasket 827 (e.g., an o-ring) that may aid in sealing the valve 800 when the distal plug 820 is engaged with the distal plug receiver 825. The valve 800 may also include a flange 830, which abuts the arms 811a and 811b. The flange 830 may serve to both hold the actuator 810 in position within the tubing 850 and provide a surface against which the resilient arms 811a and 811b are pressed so that they may flex toward the piston 815. When the gas or fluid applies a threshold pressure to the distal end of the distal plug 820, the distal plug 820 may be unseated from the plug receiver 825 as resilient flexible arms 811a and 811b are pressed against flange 830, flex toward the piston 815, and the angles between the resilient flexible arms 811a and 811b and the piston 815 are reduced.

FIG. 6B provides successive views of the valve 800 during the process of the distal plug 820 being unseated from the plug receiver 825. The gas, fluid, or other flowable material flows through end 851 toward the distal plug 820, as shown in the top view (the closed condition). As the gas, fluid, or other flowable material reaches the pre-determined threshold pressure, it pushes on the distal end of the distal plug 820 and unseats the distal plug 820 from the plug receiver 825, as shown in the bottom view (the open condition). In the open condition, the gas, fluid, or other flowable material flows through the opening 826 and the valve 800 from the upstream side 851 to the downstream side 852 of the valve 800. Once the pressure of the gas, fluid, or other flowable material falls below the pre-determined threshold pressure, the spring-like action of the resilient actuator (i.e., the resilience of the resilient arms) causes the actuator to resile to its original shape with the distal plug 820 seated in the plug receiver 825 in the closed condition.

FIG. 6C shows a further example of an in-line check valve 900 according to an embodiment of the present invention. The valve 900 may be positioned within tubing 950 for passing a gas, liquid, or other flowable material. The direction of flow is indicated by the arrows in the figure. The valve 900 includes an actuator 910 having a piston 915 and a distal plug 920 having a distally tapering shape (e.g., conical) and resilient flexible arms 911a and 911b, which may be attached at their distal ends to the interior wall of the tubing 950 at points 912a and 912b. The resilient flexible arms 911a and 911b of the actuator 910 may also be attached at their proximal ends to the proximal end of the piston at flexible joints that allow the arms to flex toward the piston. In the tubing 950, a plug receiver 925 may be formed therein having a complementary shape to the distal plug 920. The distally tapering shape of the distal plug 920 is operable to engage and obstruct the plug receiver 925, thereby preventing liquid, gas, or other flowable material from passing through the tubing 950. The distal plug receiver may include a gasket 927 (e.g., an o-ring) that may aid in sealing the valve 900 when the distal plug 920 is engaged with the distal plug receiver 925. When the gas or fluid applies a threshold pressure to the distal end of the distal plug 920, the distal plug 920 may be unseated from the plug receiver 925 as resilient flexible arms 911a and 911b may flex inward toward the piston 915, and the angles between the resilient flexible arms 911a and 911b and the piston 915 are reduced. The wall of the tubing 950 may also flex inward at points 912a and 912b to facilitate movement and deformation of the actuator 910.

FIG. 6C provides successive views of the valve 900 during the process of the distal plug 920 being unseated from the plug receiver 925. The gas, fluid, or other flowable material flows through end 951 toward the distal plug 920, as shown in the top view (the closed condition). As the gas, fluid, or other flowable material reaches the pre-determined threshold pressure, it pushes on the distal end of the distal plug 920 and unseats the distal plug 920 from the plug receiver 925, as shown in the bottom view (the open condition). Additionally, the resilient arms 911a and 912a flex toward the piston 915 and the wall of the tubing 950 flexes at points 912a and 912b. In the open condition, the gas, fluid, or other flowable material flows through the opening 926 and the valve 900 from the upstream side 951 to the downstream side 952 of the valve 900. Once the pressure of the gas, fluid, or other flowable material falls below the pre-determined threshold pressure, the spring-like action of the resilient actuator (i.e., the resilience of the resilient arms) causes the actuator to resile to its original shape with the distal plug 920 seated in the plug receiver 925 in the closed condition.

### CONCLUSION

The present invention provides a novel pinch valve structure that allows for finely controlled dispensing of liquids, gases, and other flowable materials. The present invention address issues with current valves with an innovative structure that prevents leakage and waste, and improves efficiency and ease of use.

It is to be understood that variations, permutations, and modifications of the present invention may be made without departing from the scope thereof. One or more features of an exemplary embodiment as described above may be practiced in conjunction with other exemplary embodiments as described above. It is also to be understood that the present invention is not to be limited by the specific embodiments disclosed herein, but only in accordance with the appended claims when read in light of the foregoing specification.

What is claimed:

1. A valve comprising:
a. a resilient compressible body having a distal drainage hole integrally formed in the resilient compressible body; and
b. a resilient actuator positioned within said resilient compressible body comprising:
i. at least two resilient arms, wherein each of said at least two resilient arms remains in contact with an interior wall of said resilient compressible body and said resilient actuator is maintained in its position within the compressible body by said contacts without additional connections both when said valve is in a closed condition and no external force is applied to the resilient compressive body, and when said valve is in an open condition and external force is applied to said resilient compressible body; and
ii. a central piston member mechanically connected to said at least two resilient arms, wherein each of said at least two resilient arms is attached to the central piston member at an oblique angle with respect to the longitudinal axis of the central piston member,
a distal plug connected to a distal end of said central piston member, said distal plug being operable to be inserted into and retracted from said distal drainage hole.

2. The valve of claim 1, wherein said at least two resilient arms each include an upper portion connected to said central piston member, lower portion, and a flexible elbow where said upper portion and said lower portion meet.

3. The valve of claim 1, further comprising a proximal attachment mechanism operable to connect to a vessel containing a fluid.

4. The valve of claim 1, wherein said resilient compressible body has a distal plug guide adjacent to said distal drainage hole, said distal plug guide being integral to said resilient compressible body and operable to guide and position said distal plug in said distal drainage hole.

5. (canceled)

6. The valve of claim 4, wherein said distal plug has a conical shape and said distal plug guide has a shape that is complementary to said conical shape.

7. The valve of claim 1, wherein said valve has a closed condition and an open condition, wherein
a. in said closed condition said resilient actuator is in a relaxed conformation with a first angle between each of said at least two resilient arms and said central piston member when no external force is applied to said resilient compressible body, and said distal plug is engaged with said distal drainage hole; and
b. in said open configuration said external force is applied to said resilient compressible body and said at least two resilient arms, wherein said resilient actuator is in a compressed conformation with a second angle between each of said at least two resilient arms and said central piston member, said second angle being smaller than the first angle, and said distal plug is retracted from engagement with said distal drainage hole.

8. The valve of claim 1, wherein said at least two resilient arms are mechanically connected to said interior wall of said resilient compressible body.

9. (canceled)

10. The valve of claim 1, wherein said resilient actuator includes at least three resilient arms, wherein each of said at least three resilient arms remains in contact with said interior wall of said resilient compressible body.

11. (canceled)

12. (canceled)

13. (canceled)

14. (canceled)

15. A valve comprising:
a. a resilient compressible body having a distal drainage hole; and
b. an actuator within said resilient compressible body comprising
i. at least two resilient actuator arms, wherein each of said at least two resilient arms are connected with an interior wall of said resilient compressible body to maintain said actuator in position within said compressible body; and
ii. a distal plug in mechanical communication with said at least two resilient arms and operable to be inserted into and retracted from said distal drainage hole, wherein said at least two resilient actuator arms are compressed when said compressible body is compressed by pressure applied to the exterior of compressible body and said distal plug is retracted from said distal drainage hole when said at least two resilient actuator arms are compressed.

16. The valve of claim 15, further comprising a proximal attachment mechanism operable to connect said valve to a fluid conduit or reservoir.

17. The valve of claim 15, wherein said valve has a closed configuration and an open configuration, wherein
a. in said closed configuration said resilient actuator is in a relaxed conformation with a first angle between said at least two resilient arms when no external force is applied to said resilient compressible body, and said distal plug is engaged with said distal drainage hole; and
b. in said open configuration an external force is applied to said resilient compressible body and said at least two resilient arms, wherein said resilient actuator is in a compressed conformation with a second angle between said at least two resilient arms, said second angle being smaller than the first angle, and said distal plug is retracted from engagement with said distal drainage hole.

18. The valve of claim 15, wherein the resilient actuator is coated with a non-reactive material.

19. The valve of claim 15, further comprising a distal plug guide integrally formed in said resilient compressible body at said distal drainage hole that is operable to guide and position said distal plug in said distal drainage hole.

20. The valve of claim 19, wherein said distal plug guide has a shape that is complementary to a shape of said distal plug.

21. (canceled)

22. (canceled)

23. The valve of claim 15, wherein a distal end of at least one of said at least two resilient arms is connected to said interior wall of said resilient compressible body.

24. The valve of claim 15, wherein at least one of said at least two resilient arms includes an interfacing plate at a distal end thereof, said interfacing plate being secured to said interior wall of said resilient compressible body.

25. The valve of claim 15, further comprising a resilient ring structure secured to said at least two resilient arms and in mechanical contact with said interior wall of said resilient compressible body.

26-32. (canceled)

33. A check valve comprising:
a. an actuator positioned entirely within a resilient compressible tubing for passing a gas or fluid, said actuator comprising:
i. at least two arms secured to an interior wall of said resilient compressible tubing;
ii. a central piston member mechanically connected to said at least two arms, wherein each of said at least two arms is attached to the central piston member at an oblique angle with respect to the longitudinal axis of the central piston member; and
iii. a distal plug connected to a distal end of said central piston member; and
b. a plug receiver positioned entirely within said resilient compressible tubing for receiving said distal plug, wherein said distal plug is operable to be inserted into and retracted from said plug receiver.

34. The check valve of claim 33, wherein said actuator has a pre-determined threshold resistance, such that when a gas or fluid applies a pressure equal to said pre-determined threshold resistance, the distal plug is unseated from said plug receiver and said check valve is opened.

35. (canceled)

36. The check valve of claim 33, wherein said distal plug has a conical shape and said plug receiver has a shape that is complementary to said conical shape.

37. The check valve of claim 33, wherein said check valve has a closed configuration and an open configuration, wherein
a. in said closed configuration said actuator is in a relaxed conformation with said distal plug engaged with said plug receiver; and
b. in said open configuration said actuator is in an extended conformation wherein said distal plug is retracted from engagement with said plug receiver.

38. The check valve of claim 33, wherein said at least two arms are secured to said interior wall of said resilient compressible tubing.

39. (canceled)

40. (canceled)

41. The check valve of claim 33, wherein the actuator is coated with a hydrophobic material.

42. (canceled)

43. The check valve of claim 33, wherein the actuator is coated with a non-reactive material.

44-81. (canceled)

82. The valve of claim 1, wherein said at least two resilient arms are articulably connected to said interior wall of said compressible body.

83. The valve of claim 1, wherein at least one of said at least two resilient arms are articulably connected to said central piston member.

84. The valve of claim 1, wherein said at least two resilient arms, said central piston member, and said distal plug of said actuator are integrally formed.

85. The valve of claim 15, wherein said at least two resilient arms are articulably connected to said interior wall of said compressible body.

86. The valve of claim 15, further comprising a central piston member between said distal plug and said at least two resilient arms.

87. The valve of claim 86, wherein said at least two resilient arms are articulably connected to said central piston member.

88. The valve of claim 15, wherein said at least two resilient arms and said distal plug of said actuator are integrally formed.

89. The valve of claim 15, wherein said distal drainage hole is integrally formed in said resilient compressible body.

90. The valve of claim 19, wherein said distal plug guide is integrally formed in said resilient compressible body.

91. The check valve of claim 33, wherein said at least two arms, said central piston member, and said distal plug of said actuator are integrally formed.

92. The check valve of claim 33, wherein said at least two resilient arms are articulably connected to said central piston member.

93. The check valve of claim 33, wherein said at least two resilient arms are articulably connected to said interior wall of said compressible body.

94. The check valve of claim 1, wherein contact between said resilient compressible body and said resilient actuator consists only of a single point of contact between each arm of said at least two resilient arms and said interior wall, and contact between said distal plug and said distal drainage hole.

95. The check valve of claim 15, wherein said resilient actuator contacts said resilient compressible body only where said at least two resilient arms contact said interior wall, and at the point of engagement between said distal plug and said distal drainage hole.
